(19) United States
(12) Patent Application Publication
(10) Pub. No.: US 2019/0083167 A1
(43) Pub. Date: Mar. 21, 2019

(54) MEDICAL MANIPULATOR AND END EFFECTOR UNIT

(71) Applicant: Olympus Corporation, Tokyo (JP)

(72) Inventor: Ryoji Hyodo, Tokyo (JP)

(73) Assignee: Olympus Corporation, Tokyo (JP)

(21) Appl. No.: 16/194,626

(22) Filed: Nov. 19, 2018

Related U.S. Application Data
(63) Continuation of application No. PCT/JP2016/088035, filed on Dec. 21, 2016.

(51) Int. Cl. A61B 18/14; A61B 34/00

(52) U.S. Cl. CPC A61B 18/1442; A61B 34/30; A61B 34/70

(57) ABSTRACT

The technology disclosed herein is directed to a medical manipulator having an elongated member. The elongated member includes a base member and a proximal end member on opposed ends thereof. An end effector unit is detachably attached to the base member so as to be in an electrical communication with the proximal end member. The base member is formed by a flat face surface and an outwardly projecting curved face surface extending longitudinally therefrom. The base member includes a protrusion projecting from the cured face surface in a direction perpendicular to a longitudinal axis of the elongated member. The end effector unit includes an engaging portion being used to engage with the protrusion to securely attach the end effector unit to the base member during an operation of the medical manipulator on a body tissue. The end effector is detached and discarded after the operation.

### CROSS-REFERENCE TO RELATED APPLICATION

This application is a continuation application of PCT Application No. PCT/JP2016/088035 filed on Dec. 21, 2016, which is hereby incorporated by reference in its entirety.

### TECHNICAL FIELD

The disclosed technology relates to a medical manipulator and an end effector unit.

### DESCRIPTION OF THE RELATED ART

There is known a medical manipulator with an electrode detachably mounted on one distal end thereof as disclosed in the U.S. Pat. No. 7,367,973 (hereinafter, U.S. Pat. No. 7,367,973).

When the electrode of the medical manipulator is energized in surgery, a body tissue tends to stick to the electrode and the electrode is difficult to clean off. The electrode is detachable and disposable, freeing the users or operators from the difficulty in cleaning the electrode. As noted, the electrode is disposable and thus is prevented from being damaged or deformed by a cleaning process.

However, the medical manipulator disclosed in U.S. Pat. No. 7,367,973 is disadvantageous in that the main part of the medical manipulator from which the electrode has been detached also needs to be cleaned because it is placed in the patient's body and kept in contact with a body tissue, but is difficult to clean on account of its complex structure.

It would therefore be advantageous to develop a medical manipulator having an end effector that overcome the shortcomings of the prior art.

### BRIEF SUMMARY OF EMBODIMENTS

Embodiments of the technology disclosed herein is directed to a medical manipulator and an end effector unit which make it easy to clean a main part of the medical manipulator from which an electrode has been detached and to thereby keeping the medical manipulator highly clean.

According to a first aspect of the technology disclosed herein, a medical manipulator comprises an elongated member having a base member on a distal end thereof. An end effector unit is detachably attached to the base member. A proximal end member is connected to a proximal end of the elongated member for actuating the end effector unit. The base member has a surface formed a flat face and/or an outwardly projecting curved face, and a protrusion projecting from the surface in a direction perpendicular to a longitudinal axis of the elongated member. The end effector unit has an engaging portion which engages the protrusion to keep the end effector unit undislodgeably attached to the base member when the end effector unit is attached to the base member.

With the first aspect, when the end effector unit is attached to the base member of the elongated member, the engaging portion of the end effector unit engages the protrusion of the base member to keep the end effector unit undislodgeably attached to the base member. Then, the end effector unit is actuated by the proximal end member on the proximal end of the elongated member to treat a body tissue in an area to be treated.

After the treatment has been finished, the engaging portion of the end effector unit and the protrusion of the base member are disengaged from one another, so that the end effector unit is detached from the base member. Since the surface of the base member is formed the flat face and/or the outwardly projecting curved face, it can easily be cleaned to keep the base member highly clean even if body tissues and body fluids or the likes are deposited thereon. The base member and the end effector unit may have respective contacts that are electrically connected to each other when the end effector unit is attached to the base member and the end effector unit includes a resilient member normally biasing the contact in a direction to be pressed against the base member. With this arrangement, despite the simple structure in which the engaging portion of the end effector unit engages the protrusion of the base member, the contacts of the end effector unit and the base member are reliably electrically connected to each other because the contacts are pressed against each other under the biasing force produced by the resilient member. Furthermore, the base member may be cylindrical in shape, and the end effector unit may be shaped as a cap to be fitted over the base member. The cap-shaped end effector unit is fitted over the cylindrically-shaped base member to cover the base member so as to make it less liable for body fluids and body tissues to be deposited on the base member. Moreover, the medical manipulator may further include a seal member fully circumferentially sealing a clearance between an outer circumferential surface of the base member and an inner circumferential surface of the end effector unit.

With this arrangement, when the cap-shaped end effector unit is fitted over the cylindrically-shaped base member, the seal member prevents body fluids and body tissues from entering the medical manipulator through the clearance between the base member and the end effector unit. Furthermore, the base member may be attached to a distal end of a joint, and the medical manipulator may further include a tubular cover protecting the joint. The tubular cover prevents body fluids and body tissues from being deposited on the joint.

According to a second aspect of the technology disclosed herein, an end effector unit is detachably attached to a base member of a medical manipulator. The medical manipulator includes an elongated member, a proximal end member connected to a proximal end of the elongated member, and the base member disposed on a distal end of the elongated member. The base member includes a surface formed a flat face and/or an outwardly projecting curved face and a cylindrical protrusion projecting from the surface in a direction perpendicular to a longitudinal axis of the elongated member. The end effector unit includes an engaging portion which engages the cylindrical protrusion to keep the end effector unit undislodgeably attached to the base member when the end effector unit is attached to the base member.

In the second aspect, the end effector unit may further include a contact electrically connected when the end effector unit is attached to the base member, and a resilient member normally biasing the contact in a direction to be pressed against the base member. Furthermore, the end effector unit may be shaped as a cap to be fitted over the base member. Moreover, the end effector unit may further include a seal member disposed in an inner circumferential surface thereof to be fitted over the base member and sealing a clearance created when the end effector unit is fitted over the base member. Accordingly, it is easy to clean a main part of the medical manipulator from which an electrode has been detached and thereby keeping the medical manipulator highly clean.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a plan view of a medical manipulator according to an embodiment described herein.

FIG. 2 is a fragmentary perspective view of the medical manipulator depicted in FIG. 1 illustrating an end effector unit on a distal end of the medical manipulator.

FIG. 3 is a fragmentary perspective view of the medical manipulator depicted in FIG. 1 illustrating the manner in which an end effector unit is detached from a base member depicted in FIG. 2.

FIG. 4 is a perspective view of the end effector unit according to the embodiment described herein.

FIG. 5 is a fragmentary cross-sectional view of the medical manipulator depicted in FIG. 3 taking along line 5-5 depicting the manner in which the end effector unit is detached from the base member of the medical manipulator.

FIG. 6 is a fragmentary cross-sectional view depicting the manner in which the end effector unit is attached to the base member of the medical manipulator depicted in FIG. 5.

FIG. 7 is a fragmentary side elevational view depicting the manner in which the end effector unit is detached from the base member of the medical manipulator depicted in FIG. 1.

FIG. 8 is a fragmentary side elevational view depicting the manner in which the end effector unit is attached to the base member of the medical manipulator depicted in FIG. 1.

FIG. 9 is a fragmentary cross-sectional view taking along line 9-9 in FIG. 1 depicting the manner in which an end effector unit is detached from a base member according to a first modification of the medical manipulator depicted in FIG. 1.

FIG. 10 is a fragmentary cross-sectional view depicting the manner in which the end effector unit is attached to the base member of the medical manipulator depicted in FIG. 9.

FIG. 11 is a fragmentary cross-sectional view of a medical manipulator according to a second modification of the embodiment depicted in FIG. 1.

FIG. 12 is a fragmentary perspective view of a medical manipulator according to a third modification of the embodiment depicted in FIG. 1.

FIG. 13 is a fragmentary perspective view of a medical manipulator according to a fourth modification of the embodiment depicted in FIG. 1.

FIG. 14 is a fragmentary perspective view of a medical manipulator according to a fifth modification of the embodiment depicted in FIG. 1.

### DETAILED DESCRIPTION OF THE EMBODIMENTS

A medical manipulator 1 and an end effector unit 3 according to an embodiment of the technology disclosed herein is described hereinbelow with reference to the drawings.

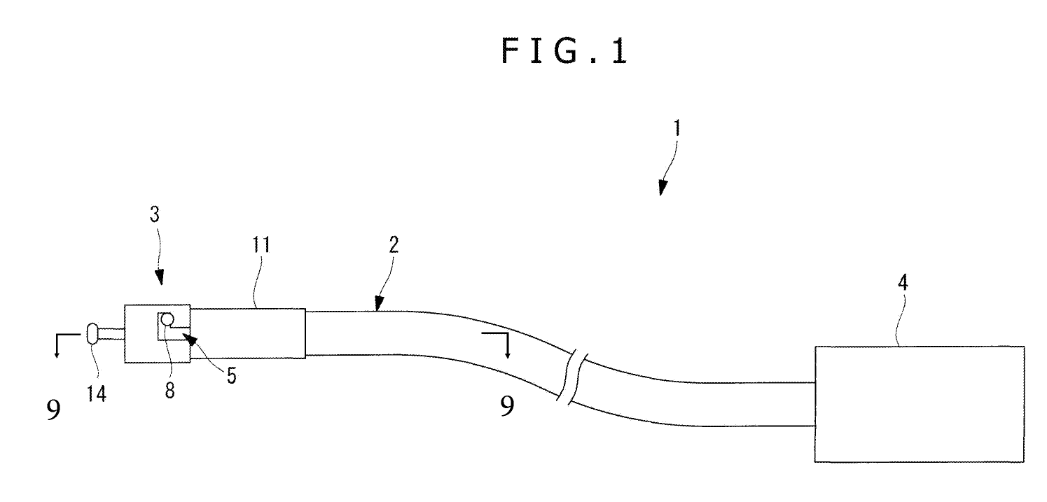

As depicted in FIG. 1, the medical manipulator 1 includes an elongate insertion portion or elongated member 2 and the end effector unit 3 that is disposed on a distal end of the insertion portion 2. A proximal end member 4 is disposed on a proximal end of the insertion portion 2 for actuating the end effector unit 3.

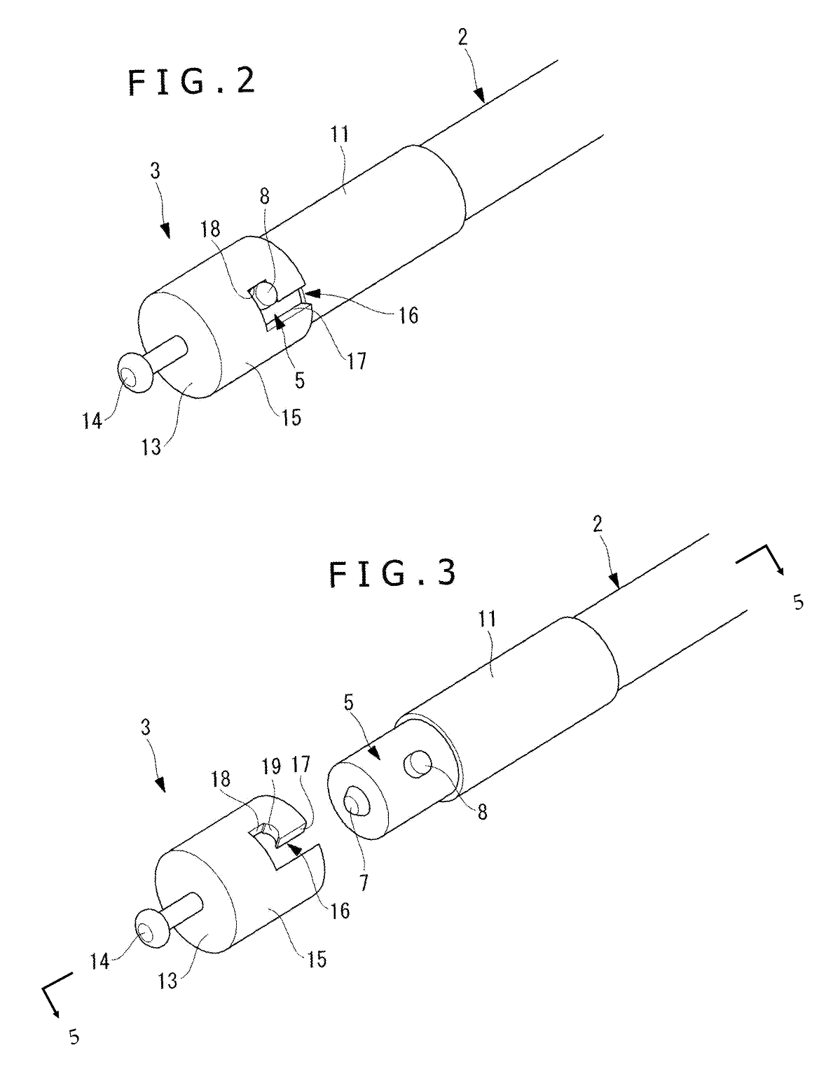

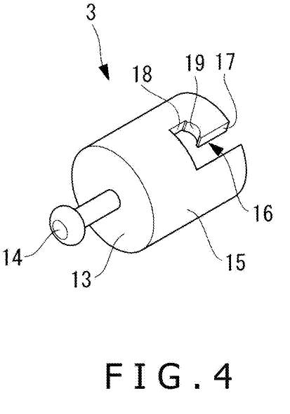

The elongated member or elongate insertion portion 2 includes a base member 5 on its distal end for attaching to the end effector unit 3 thereon. As depicted in FIGS. 2 through 4, the end effector unit 3 is detachably attached to the base member 5 so that it can be removed therefrom and can be installed thereon along the longitudinal directions of the insertion portion 2. The base member 5 is of a cylindrical shape and has a contact 7 exposed on a distal end face thereof. The contact 7 is projected outwardly along the longitudinal direction of the insertion portion 2. The base member 5 also has a cylindrical protrusion 8 that projects radially outwardly from a side surface thereof. The base member 5 thus has its outer face or surface formed a flat face and/or an outwardly projecting curved face. In other words, the base portion 5 does not include concave surface so that the base portion 5 is easy to be cleaned.

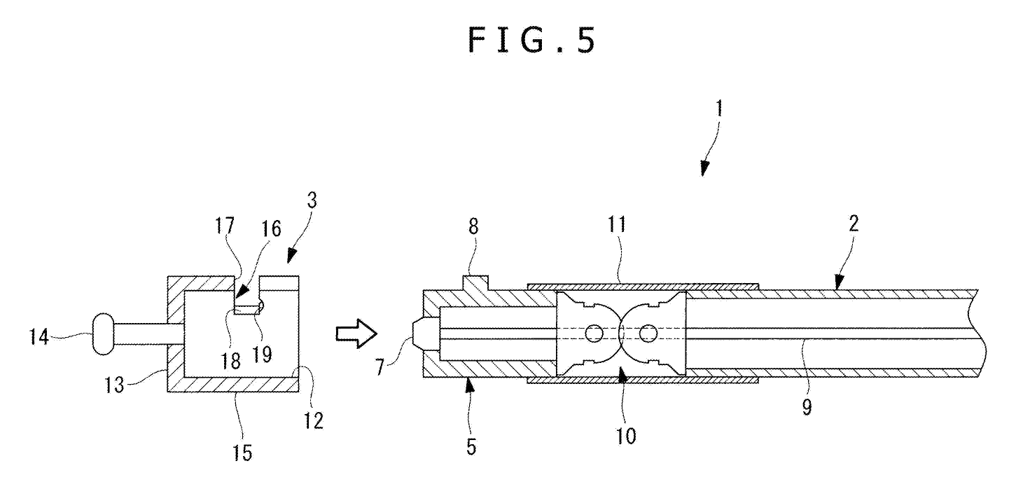

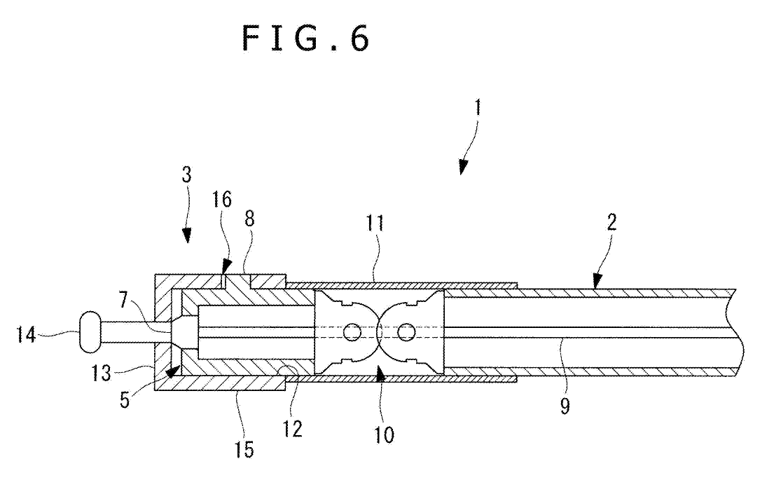

As depicted in FIGS. 5 and 6, the contact 7 exposed on the distal end face of the base member 5 is connected to an electric cable 9 that extends longitudinally in the base member 5. The electric cable 9 also extends longitudinally in the elongate insertion portion 2 and is connected to the proximal end member 4 disposed on the proximal end of the elongate insertion portion 2.

The elongate insertion portion 2 houses in its distal end a joint 10 that allows the base member 5 to swing about an axis perpendicular to the longitudinal axis of the elongate insertion portion 2. The joint 10 has the electric cable 9 extending therethrough and is actuatable by the proximal end member 4 through power transmitting members such as wires, etc., not depicted.

The joint 10 is covered with a tubular cover 11 fitted over an outer circumferential surface of the elongate insertion portion 2 near a proximal end of the joint 10 and an outer circumferential surface of the base member 5 near a distal end of the joint 10. With tubular cover 11, the medical manipulator 1 is of a liquid-tight structure that prevents external liquids such as body fluids or the like from entering all the components from the elongate insertion portion 2 to the base member 5.

The proximal end member 4 has a supply unit (not depicted) for supplying electric power and mechanical power. In response to an operation input from the user or operator, the supply unit supplies mechanical power for actuating the joint 10 to the power transmitting members, and also supplies electric power to the electric cable 9, from which the electric power will be supplied from an electrode 14, to be described later, to a body tissue.

The end effector unit 3 is shaped as a cap including a hollow cylinder 15 that has a cavity or hole 12 defined therein into which the base member 5 is to be fitted and also has an end closed by an end plate 13. The bar-shaped electrode 14 projects outwardly from the end plate 13 in a longitudinal direction of the end effector unit 3. The electrode 14 extends through the end plate 13 in a thicknesswise direction thereof and is connected to the contact member 7 that is projected outwardly.

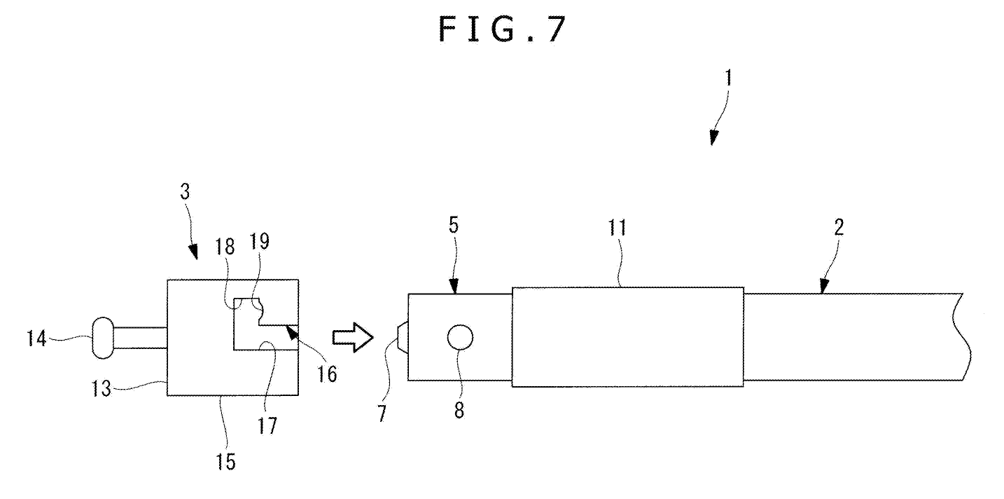

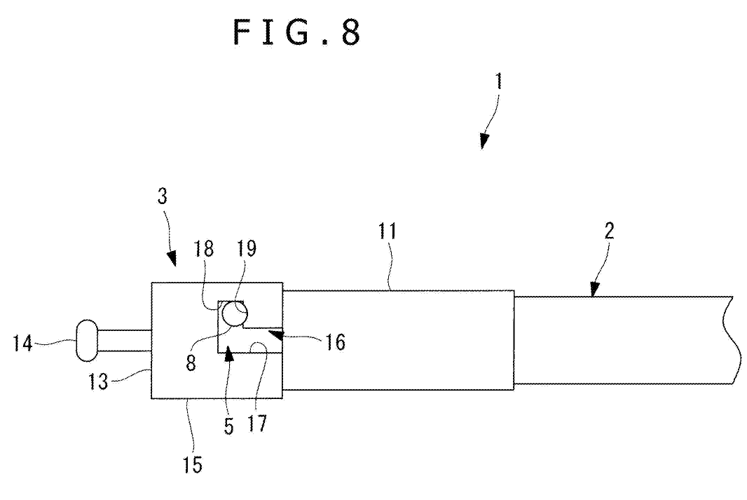

As depicted in FIGS. 7 and 8, the end effector unit 3 includes a generally L-shaped groove 16 formed on the surface of the hollow cylinder 15 which extends radially through the hollow cylinder 15 toward the end plate 13 for accommodating, guiding, and locking therein the protrusion 8 of the base member 5. The L-shaped groove 16 is released to the proximal end of the end effector unit 3 and includes an introductory portion 17 extending in a longitudinal direction from the proximal end of the end effector unit 3 and an engaging portion 18 bent from the introductory portion 17 at a middle position in the longitudinal direction and extending in a circumferential direction. An engaging recess 19 is defined in the vicinity of the terminal end of the engaging portion 18 by making an edge of the engaging portion 18 slightly recessed toward the proximal end of the end effector unit 3. The engaging recess 19 is shaped so as to be able to accommodate part of the protrusion 8 in a locking position. When the protrusion 8 is guided through the introductory portion 17 and the engaging portion 18 and engages in the engaging recess 19, the protrusion 8 is locked against removal from the engaging portion 18.

Operation of the medical manipulator 1 and the end effector unit 3 according to the technology disclosed herein will be described hereinafter.

For treating an affected area in the body of a patient (not shown) using the medical manipulator 1 according to the present embodiment, the end effector unit 3 is installed on the base member 5, and the elongate insertion portion 2 is inserted with its distal end positioned ahead into the body of the patient. The proximal end member 4 supplies mechanical power through the power transmitting members to the joint 10, setting the electrode 14 to a desired position and direction. Then, the proximal end member 4 supplies electric power through the electric cable 9 to the electrode 14, which operates the joint 10 and causes the electrode 14 to cauterize the body tissue of the affected area. When the electrode 14 cauterizes the body tissue, part of the body tissue sticks to the electrode 14 over time. It is necessary to remove the body tissue deposited on the electrode 14.

According to the present embodiment, the end effector unit 3 with the electrode 14 is detached from the base member 5 and discarded. The base member 5 is cleaned, and a new end effector unit 3 is installed on the base member 5, so that the medical manipulator 1 can be used again. For detaching the end effector unit 3 from the base member 5, an external force for turning the end effector unit 3 with respect to the base member 5 about their longitudinal axis is applied to dislodge the protrusion 8 out of the engaging recess 19 of the groove 16 and turn the end effector unit 3 with respect to the base member 5 about their longitudinal axis.

The protrusion 8 moves in the groove 16 along the engaging portion 18 into the introductory portion 17, and then the end effector unit 3 is moved away from the base member 5 along their longitudinal axis, displacing the protrusion 8 along the introductory portion 17. The end effector unit 3 can thus easily be detached from the base member 5.

It should be noted that the construction of the medical manipulator 1 affords significant advantage with respect to protecting the manipulator 1 against any foreign materials such as body fluids or the like during operation. Particularly, since the base member 5 has its outer face formed the flat face and the outwardly projecting face, and the joint 10 is disposed at the proximal end of the base member 5 is covered with the tubular cover 11 in a liquid-tight manner, then the body fluids that is deposited can simply and reliably be removed by cleaning.

As a result, the electrode 14 does not need to be cleaned, or specifically it is not necessary to perform a cleaning process for scraping off the body fluids or body tissues that have stuck to the electrode 14 with a brush or the like, since the electrode is discarded. Another advantage is that the main parts of the base member 5 and the joint 10, from which the end effector unit 3 with the electrode 14 has been detached and discarded, can easily be cleaned and hence can be kept highly clean.

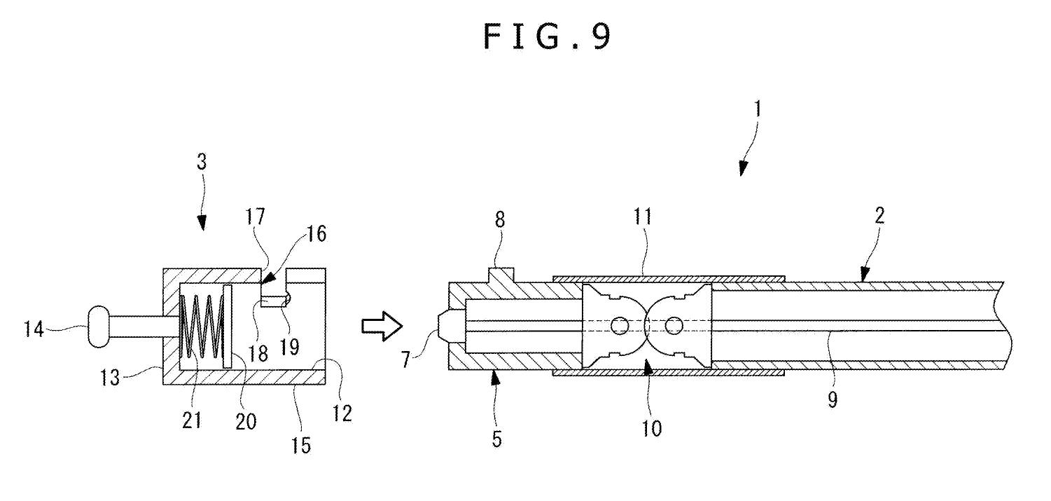

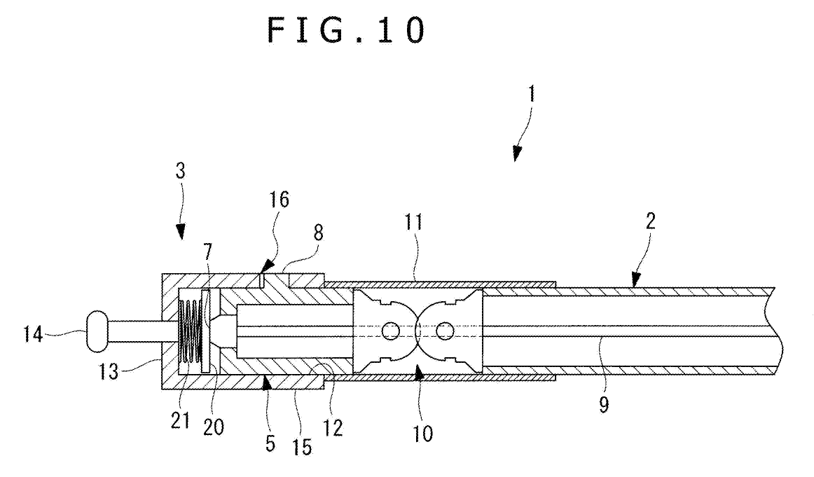

In the present embodiment, as depicted in FIGS. 9 and 10, a contact member 20 movable longitudinally is provided in the end effector unit 3, and a helical spring (resilient member) 21 made of an electrically conductive material for normally biasing the contact member 20 toward the proximal end is disposed between the contact member 20 and the electrode 14. The helical spring 21 is configured to stabilize contact pressure between the contact member 20 and the contact 7 on the base member 5 so as to avoid a contact failure therebetween. The biasing force of the helical spring 21 is effective to keep the protrusion 8 engaging in the engaging recess 19 in a simple and reliable manner.

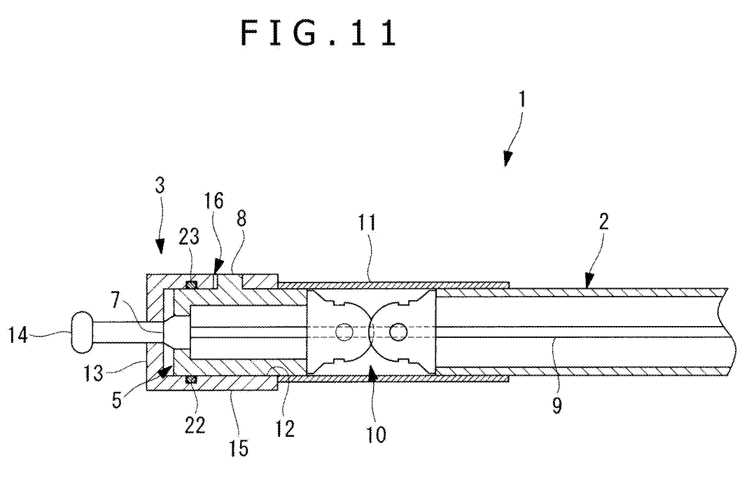

As depicted in FIG. 11, the end effector unit 3 may include a fully circumferential groove 22 defined in an inner circumferential surface thereof and a seal member 23 such as an O-ring or the like is placed in the circumferential groove 22. With this arrangement, the formation of a recess or concave surface in an outer circumferential surface of the base member 5 is avoided, and a clearance between the outer circumferential surface of the base member 5 and the inner circumferential surface of the end effector unit 3 is sealed in a liquid-tight manner. During the operation, body fluids or the like are thus prevented from finding their way into a region where the contact 7 and the electrode 14 are held in contact with each other.

In the description of the disclosed embodiment, the groove 16 extends radially through the hollow cylinder 15 of the end effector unit 3. However, the groove 16 may be defined in an inner circumferential surface of the end effector unit 3. Though the groove 16 has been illustrated as being of an L shape, it may instead be of any of other geometrical shapes and the joint 10 may have a single axis or may have a plurality of axes. The base member 5 is illustrated as being of a cylindrical shape and the end effector unit 3 has been described as being a hollow cylinder. However, the base member 5 may instead be of any geometrical shapes insofar as it has a surface formed a flat face and an outwardly projecting curved face.

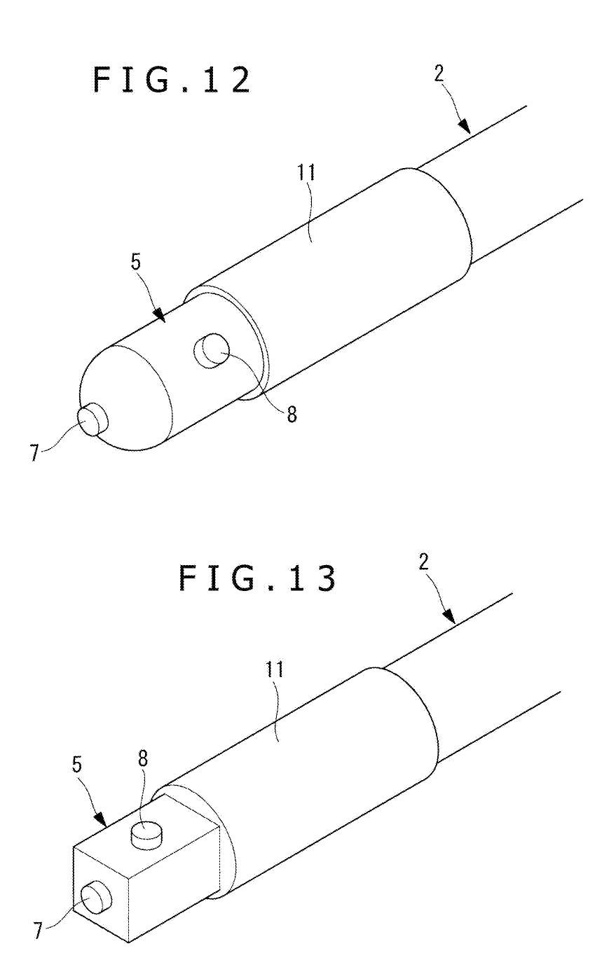

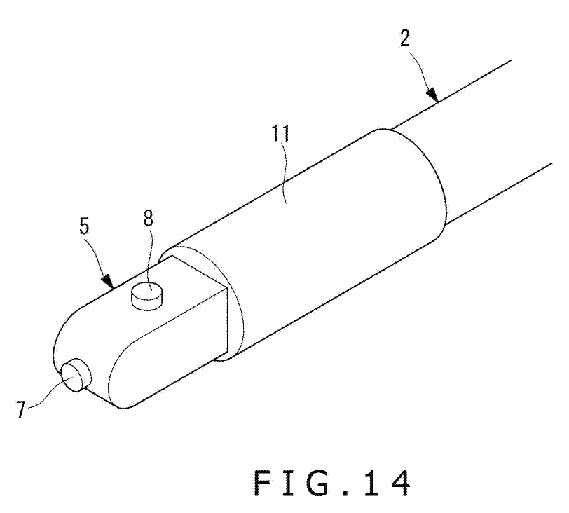

In the description of the disclosed embodiment, the base member 5 has a surface formed a flat face and an outwardly projecting curved face. However, the base member 5 may be of another shape having a surface formed a flat face and/or an outwardly projecting curved face. For example, as depicted in FIG. 12, the base member 5 may have a surface formed of a projecting curved face on its distal end and an outwardly projecting curved face. Alternatively, as depicted in FIG. 13, the base member 5 may have a surface formed flat faces, or as depicted in FIG. 14, the base member 5 may have a surface formed of a projecting curved face on its distal end and flat faces. Furthermore, the aforementioned surface may be beveled or polished as well.

With the aforementioned structures, it is possible to eliminate curved faces that are projected toward the proximal end (curved faces that are recessed toward the distal end) and/or faces having recessed portions that are projected inwardly where body tissues and body fluids, etc. are likely to be deposited. In other words, the base member 5 is securely remained clean.

In sum, one aspect of the disclosed technology is directed to the a medical manipulator comprises an elongated member having a base member and a proximal end member on opposed ends thereof the elongated member comprises a tubular cover used to securely seal the joint. An end effector unit is configured to be detachably attached to the base member so as to be in an electrical communication with the proximal end member. The base member is formed by a flat face surface and an outwardly projecting curved face surface extending longitudinally therefrom. The base member includes a protrusion projecting from the curved face surface in a direction perpendicular to a longitudinal axis of the elongated member. The end effector unit includes an engaging portion being used to engage with the protrusion to securely attach the end effector unit to the base member during an operation of the medical manipulator. The end effector is detached and discarded after the operation.

The base member includes a contact member and the end effector unit includes an electrode that is electrically connected to the contact member. The base member is cylindrical in shape and the end effector unit is a cylindrical cap used to be fitted over said the base member. The end effector unit includes a resilient member normally biasing the contact member in a direction to be pressed against the base member. A seal member is configured to circumferentially sealing a clearance between an outer circumferential surface of the base member and an inner circumferential surface of the end effector unit. The base member is attached to a distal end of a joint.

Another aspect of the disclosed technology is directed to a medical manipulator defined by an elongated member having a base member on one end and a proximal end member on an opposed end. An end effector unit is detachably attached to the base member. The end effector unit comprises an elongated end cap having an enclosed first end and an open second end. An electrode is configured to be projected outwardly from the enclosed first end. A groove is formed radially from the open second end extending toward the enclosed first end so that the groove being used to be engaged with the base member. The groove is defined by an introductory portion extending in a longitudinal direction from the open second end and an engaging portion bent from the introductory portion at a middle position and extending in a circumferential direction forming an L-shaped groove. The end effector unit includes a contact member movable longitudinally and a helical spring for biasing the contact member toward the proximal end member. The helical spring is used to stabilize contact pressure between the contact member and a contact on the base member so as to avoid a contact failure therebetween. A seal member is configured to circumferentially sealing a clearance between an outer circumferential surface of the base member and an inner circumferential surface of the end effector unit.

A further aspect of the disclosed technology is directed to an end effector unit being detachably attached to a medical manipulator. The end effector unit comprises an elongated end cap having an enclosed first end and an open second end. An electrode is configured to be projected outwardly from the enclosed first end. A groove is formed radially from the open second end extending toward the enclosed first end so the groove is used to engage with the medical manipulator. The groove is defined by an introductory portion extending in a longitudinal direction from the open second end and an engaging portion bent from the introductory portion at a middle position and extending in a circumferential direction forming an L-shaped groove.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional,” “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A medical manipulator comprising:
an elongated member having a base member and a proximal end member on opposed ends thereof; and
an end effector unit configured to be detachably attached to the base member so as to be in an electrical communication with the proximal end member, and
wherein the base member being formed by a flat face surface and an outwardly projecting curved face surface extending longitudinally therefrom, the base member includes a protrusion projecting from the curved face surface in a direction perpendicular to a longitudinal axis of the elongated member, and
the end effector unit includes an engaging portion being used to engage with the protrusion to securely attach the end effector unit to the base member during an operation of the medical manipulator and wherein the end effector being detached and discarded after the operation.

2. The medical manipulator of claim 1,
wherein the base member includes a contact member and the end effector unit includes an electrode that is electrically connected to the contact member and
wherein the end effector unit includes a resilient member normally biasing the contact member in a direction to be pressed against the base member.

3. The medical manipulator of claim 1
wherein the base member is cylindrical in shape and the end effector unit is a cylindrical cap used to be fitted over said the base member.

4. The medical manipulator of claim 3 further comprising:
a seal member being configured to circumferentially sealing a clearance between an outer circumferential surface of the base member and an inner circumferential surface of the end effector unit.

5. The medical manipulator of claim 1,
wherein the base member is attached to a distal end of a joint.

6. The medical manipulator of claim 5, wherein the elongated member comprises a tubular cover used to securely seal the joint.

7. A medical manipulator defined by an elongated member having a base member on one end and a proximal end member on an opposed end and an end effector unit being detachably attached to the base member wherein the end effector unit comprising:
an elongated end cap having an enclosed first end and an open second end,
an electrode configured to be projected outwardly from the enclosed first end, and
a groove being formed radially from the open second end extending toward the enclosed first end so that the groove being used to be engaged with the base member and wherein
the groove being defined by an introductory portion extending in a longitudinal direction from the open second end and an engaging portion bent from the introductory portion at a middle position and extending in a circumferential direction forming an L-shaped groove.

8. The medical manipulator of claim 7, wherein the end effector unit includes a contact member movable longitudinally and a helical spring for biasing the contact member toward the proximal end member.

9. The medical manipulator of claim 7, wherein the helical spring is used to stabilize contact pressure between the contact member and a contact on the base member so as to avoid a contact failure therebetween.

10. The medical manipulator of claim 7 wherein the base member is cylindrical in shape and the end effector unit is a cylindrical cap used to be fitted over said the base member.

11. The medical manipulator of claim 10 further comprising a seal member being configured to circumferentially sealing a clearance between an outer circumferential surface of the base member and an inner circumferential surface of the end effector unit.

12. An end effector unit being detachably attached to a medical manipulator, the end effector unit comprising:
an elongated end cap having an enclosed first end and an open second end,
an electrode configured to be projected outwardly from the enclosed first end, and
a groove being formed radially from the open second end extending toward the enclosed first end so the groove being used to be engaged with the medical manipulator and wherein
the groove being defined by an introductory portion extending in a longitudinal direction from the open second end and an engaging portion bent from the introductory portion at a middle position and extending in a circumferential direction forming an L-shaped groove.

13. The end effector unit of claim 12, wherein medical manipulator is defined by an elongated member having a base member on one end and a proximal end member on an opposed end wherein the end effector unit is detachably attached to the base member.

14. The end effector unit of claim 13, wherein the medical manipulator includes a seal member being configured to circumferentially sealing a clearance between an outer circumferential surface of the base member and an inner circumferential surface of the end effector unit.
